SEQUENCE LISTING (26 sequences)

SEQ ID NO: 1 - DNA, 327 bp, Escherichia coli
atgaagtggcttttattaatcacattgagtctatattcttttattgtgcaaagtgctccctgtggattgaccaatgtcggggaacaacgtggaacatatatactcaaaccactgagtatgaaaggtaatctcaccgccgaaaaaattgaaaatactgggaatatcattgagtgtgcagacttgcaagagacgaccaacgccattaaagttcattttttaacacagaacgctttaaggtttaaagtcggaagtcgtcattatattattaatttcccctttgaatctgacggtaaaaaaaatagcgcttccagggaagtcatcttataa

SEQ ID NO: 2 - protein, 108 aa, Escherichia coli
MKWLLLITLSLYSFIVQSAPCGLTNVGEQRGTYILKPLSMKGNLTAEKIENTGNIIECADLQETTNAIKVHFLTQNALRFKVGSRHYIINFPFESDGKKNSASREVIL

SEQ ID NO: 3 - DNA, 69 bp, artificial
  synthetic cloning primer
ggaatggtgaaatttatcgcagatagcatttcttctaaattataagatgtgtaggctggagctgcttcg

SEQ ID NO: 4 - DNA, 64 bp, artificial
  synthetic cloning primer
ctcataaattaaattaataaaaattcatcaacttcaaggactgataatatgaatgtcctcctta

SEQ ID NO: 5 - DNA, 64 bp, artificial
  synthetic cloning primer
cgatgaaaacaaaacatatatatgccagtaaaaggagtttactgtgtaggctggagctgcttcg

SEQ ID NO: 6 - DNA, 62 bp, artificial
  synthetic cloning primer
gtatggaatggtgaaatttatcgcagatagcatttcttctaaacatatgaatatcctcctta

SEQ ID NO: 7 - DNA, 57 bp, artificial
  synthetic cloning primer
catagtaaaatcgccgcgagataacaggaaaaagtcgtgtaggctggagctgcttcg

SEQ ID NO: 8 - DNA, 55 bp, artificial
  synthetic cloning primer
tgtgattaataaaagccacttcatagtaaactccttcatatgaatatcctcctta

SEQ ID NO: 9 - DNA, 57 bp, artificial
  synthetic cloning primer
gagtgaccttccataccagttaattaaggatgatttgtgtaggctggagctgcttcg

SEQ ID NO: 10 - DNA, 55 bp, artificial
  synthetic cloning primer
aaataataccggtagcagagctaccggcaaaaaacgcatatgaatatcctcctta

SEQ ID NO: 11 - DNA, 61 bp, artificial
  synthetic cloning primer
taaggctaacctgactgcacagatcaacaaactggcttaagtgtaggctggagctgcttcg

SEQ ID NO: 12 - DNA, 61 bp, artificial
  synthetic cloning primer
taaaagaatatagactcaatgtgattaataaaagccacttcacatatgaatatcctcctta

SEQ ID NO: 13 - DNA, 30 bp, artificial
  artificial cloning primer
catgaagcttatgacagatggtatctcaac

SEQ ID NO: 14 - DNA, 27 bp, artificial
  synthetic cloning primer
cgcgagatcttagttgtacattaccac

SEQ ID NO: 15 - DNA, 28 bp, artificial
  synthetic cloning primer
catgaagcttatggcgataaatttcacc

SEQ ID NO: 16 - DNA, 27 bp, artificial
  synthetic cloning primer
cgcgagatcttagttgtacattaccac

SEQ ID NO: 17 - DNA, 28 bp, artificial
  synthetic cloning primer
catgaagcttatgaagtggcttttatta

SEQ ID NO: 18 - DNA, 27 bp, artificial
  synthetic cloning primer
cgcgagatcttataagatgacttccct

SEQ ID NO: 19 - DNA, 28 bp, artificial
  synthetic cloning primer
catgaagcttatgttaaggacgacacga

SEQ ID NO: 20 - DNA, 27 bp, artificial
  synthetic cloning primer
cgcgagatcttactggcatatatatgt

SEQ ID NO: 21 - DNA, 27 bp, artificial
  synthetic cloning primer
catgaagcttgtgagaataataacagc

SEQ ID NO: 22 - DNA, 29 bp, artificial
  synthetic cloning primer
cgcgagatcttatctcgcggagattttac

SEQ ID NO: 23 - DNA, 29 bp, artificial
  synthetic cloning primer
catgaagcttatgaaaaagtatattattc

SEQ ID NO: 24 - DNA, 27 bp, artificial
  synthetic cloning primer
cgcgagatcttaatctgcactgatttt

SEQ ID NO: 25 - DNA, 30 bp, artificial; identical to SEQ ID NO: 13
  synthetic cloning primer

SEQ ID NO: 26 - DNA, 27 bp, artificial
  synthetic cloning primer
cgcgagatcttaatcttgatgagatat

(19) United States
(12) Patent Application Publication
(10) Pub. No.: US 2011/0159593 A1
(43) Pub. Date: Jun. 30, 2011

(54) METHOD OF REDUCING MOTILITY IN BACTERIA BY OVEREXPRESSION OF A GENE OF OI-1 GENOMIC ISLAND

(76) Inventors: Brian Coombes, Dundas (CA); Uma Silphaduang, Waterloo (CA); Mohamed Karmali, Toronto (CA)

(21) Appl. No.: 12/994,545

(22) PCT Filed: Jun. 1, 2009
(86) PCT No.: PCT/CA09/00716

§ 371 Date: Mar. 10, 2011

Related U.S. Application Data
(60) Provisional application No. 61/057,305, filed on May 30, 2008.

(51) Int. Cl. C12N 15/63

(52) U.S. Cl. 435/471

(57) ABSTRACT

Z0021 was identified as a gene within the O-Island 1 (OI-1) genomic island of verocyto-toxin-producing E. coli O157:H7. The gene negatively regulates swimming motility and this decreased motility is the result of a reduction in surface expression of flagella. Overexpression of Z0021 suppresses the motility and surface expression of flagella in E. coli O157:H7, as well as in other Enterobacteriaceae including E. coli K12 and S. typhimurium SL1344. Disclosed is a method of reducing motility in bacterium by increasing the level of the peptide having the amino acid sequence as set forth in SEQ ID No:2 which is encoded by the Z0021 gene having the nucleotide sequence as set forth in SEQ ID No: 1.

### PRIOR APPLICATION INFORMATION

The instant application claims the benefit of U.S. Provisional Patent Application 61/057,305, filed May 30, 2008.

### BACKGROUND OF THE INVENTION

Verocytotoxin-producing Escherichia coli (VTEC) are food-borne pathogens that cause major outbreaks of bloody diarrhea and haemolytic-uraemic syndrome (HUS) worldwide.

VTECs have been classified in five ‘seropathotypes’ based on their relative incidence and their association with epidemic and/or serious diseases such as HUS.

Seropathotype A (serotypes O157:H7 and O157:NM) is associated with the most frequent outbreaks and severe diseases like HUS.

Previously, it has been shown that four fimbrae-encoding OIs (OI-1, OI-47, OI-141 and OI-154) were consistently present in VTEC seropathotype A strains but are absent in less virulent seropathotype B, C, D and E strains.

### SUMMARY OF THE INVENTION

According to a first aspect of the invention, there is provided a method of reducing motility in a bacterium comprising:

reducing surface expression of flagella in the bacterium by providing increased levels of a peptide having an amino acid sequence as set forth in SEQ ID No. 2, thereby reducing motility of the bacterium compared to a bacterium of similar type having normal levels of said peptide.

### BRIEF DESCRIPTION OF THE DRAWINGS

FIG. 1. Genetic organization of O-Island 1. (A) The genetic context of OI-1 shown to scale with numbers referring to nucleotide position from the leftmost end of the island. Arrows refer to open reading frames. Colour key for putative function of open reading frames (based on BLAST similarity) is shown below the figure. (B) Organization of knockout mutants produced in this work. Strain designations are shown to the left of black arrows depicting the regions chromosomal DNA deletions.

FIG. 2. Systematic deletion analysis of OI-1. Motility assays were conducted for ΔZ0021 and the trans-complemented mutant (pZ0021) in absence (A) or presence (B) of IPTG to induce the expression of plasmid-encoded Z0021 (when present). (C) Migration distance in motility agar plates was quantified and expressed as the mean radius of migration from the point of inoculation. Data are the means with standard errors from three independent experiments. (D) Motility profiles for the comprehensive OI-1 gene deletion set. Strains are indicated in the figure and swimming motility was quantified in (E). Data are the means with standard errors from three independent experiments.

FIG. 3. Single-gene complementation analysis of ΔOI-1. (A) A complete deletion of OI-1 was created as described in Methods and tested for motility. Shown is a representative experiment performed at least five times. (B) Complementation of ΔOI-1 with individual genes coded in the island. Complementation strains were tested for swimming motility on motility agar. Shown is a representative experiment performed three times. (a, wt; b, ΔOI-1(pOI-1); c, ΔOI-1(pZ0025); d, ΔOI-1(pZ0024); e, ΔOI-1(pZ0023); f, ΔOI-1(pZ0022); g, ΔOI-1(pZ0021); h, ΔOI-1(pZ0020); i, ΔOI-1(pFLAG); j, ΔOI-1). (C) Motility zones were quantified and plotted as the mean with standard error from three experiments.

FIG. 4. Deletion of Z0021 increases steady-state flagellin and surface flagella. (A) Western blot detection of flagellin (FliC) from E. coli O157:H7 EDL933, ΔOI-1 and ΔZ0021. Asterisks indicate non-specific cross-reacting bands useful as a loading control. (B through D), Transmission electron micrographs of E. coli O157:H7 EDL933 and deletion mutants. Cells were harvested from motility agar after 6 hr at 37° C. and negative stained with 0.1% uranyl acetate and viewed with a TEM. (B B′) wild-type E. coli O157:H7 EDL933, (C C′) ΔOI-1, (D D′) ΔZ0021.

FIG. 5. Heterologous transfer of Z0021 into other bacteria represses motility and produces aflagellate cells. Salmonella enterica serovar Typhimurium strain SL1344 and Escherichia coli K12 strain MG1655 were transformed with pZ0021 and induced to express Z0021 (A) or left uninduced (B). Motility was visualized (A, B) and quantified (C) from motility agar plates. Data are the means with standard error from three experiments. (D through G), Transmission electron micrographs of wild type S. Typhimurium and E. coli K12 and cells expressing Z0021. Strain identity is shown in the top right of each micrograph.

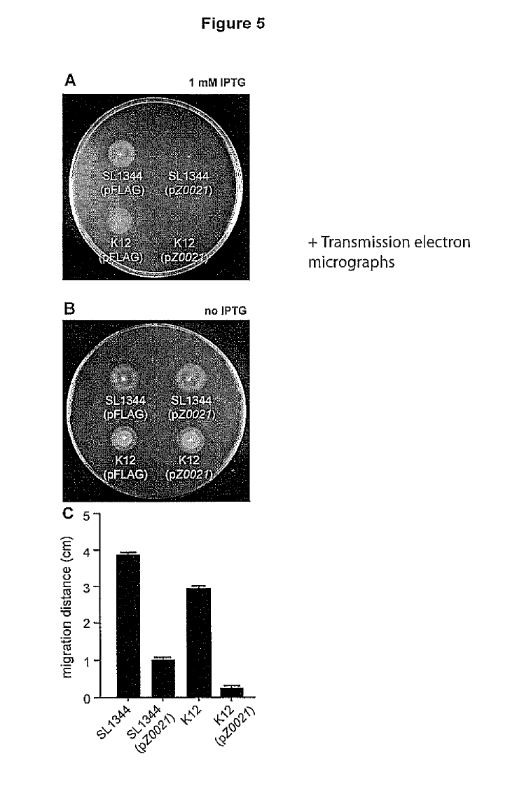

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Verotoxin-producing Escherichia coli (VTEC) are food-borne pathogens that cause major outbreaks of bloody diarrhoea, and haemolytic-uraemic syndrome worldwide. We previously reported that O-island 1 (OI-1), a genomic island containing a fimbrial operon, is highly specific for VTEC serotype O157 strains. To elucidate the function and pathogenic significance of OI-1, lambda red recombination was used to generate an OI-1 deletion mutant (ΔOI-1). We report that deletion of OI-1 resulted in significantly increased swimming motility compared to the wild-type strain, Escherichia coli EDL933. Further mutagenesis revealed that the specific gene responsible for this phenotype was Z0021, an OI-1 gene encoding a protein of unknown function in E. coli O157:H7. Complementation experiments with overexpression of the OI-1 genes, Z0021, Z0022 (usher protein) and Z0024 (type-1 fimbrial protein), resulted in a predictable loss of swimming motility. Elevated levels of flagellin in ΔOI-1 and ΔZ0021 compared to EDL933 were demonstrated by western blot analysis and enhanced surface expression of flagella was demonstrated by transmission electron microscopy (TEM). Hence, the increased motility in ΔOI-1 and ΔZ0021 is associated with the higher expression of the flagella filament suggesting a molecular connection between flagella and fimbrial formation in E. coli serotype O157. Overexpression of Z0021 protein using a plasmid encoding Z0021 in other Enterobacteriaceae including E. coli K12 MG1655 and Salmonella enterica serovar Typhimurium lead to a non-motile phenotype and loss of surface flagella. We have identified a new genetic locus in OI-1 that controls swimming motility in O157:H7 VTEC. E. coli O157 use these novel genes on the OI-1 genomic island to regulate flagellum formation and swimming motility through a novel mechanism.

As discussed herein, it has been discovered that Z0021 negatively regulates swimming motility and this decreased motility is the result of a reduction in surface expression of flagella. This implies that Z0021 may coordinate a phenotype switch between extracellular motility and adhesion. As discussed herein, overexpression of Z0021 in other Enterobacteriaceae including K12 and SL1344 also suppresses their motility and surface expression of flagella. Thus, the effect of Z0021 on motility is not exclusive to E. coli O157:H7.

While not wishing to be bound to a specific theory or hypothesis, the inventors believe that Z0021 protein binds to a protein complex comprising FIhD and FIhC proteins, based on biochemical and molecular protein interaction studies. FIhD and FIhC proteins form a complex (called FIhDC) that acts as a transcription factor to activate gene expression in the flagella expression cascade. By interrupting the FIhDC complex through a protein-protein interaction, the inventors believe that Z0021 prevents the downstream gene activation necessary to assemble flagella on the surface of the cell. The FIhDC activation pathway is conserved in E. coli K12 and Salmonella SL1344 which is why Z0021 expression in these cells has the same effect as in E. coli O157:H7 and will have the same effect in any bacterial strain in which the FIhDC pathway is conserved, that is, in a FIhDC bacterial strain, for example but by no means limited to (DON'T NEED AN EXHAUSTIVE LIST HERE—I JUST WANT TO LIST OFF THE IMPORTANT ONES AS A FALLBACK POSITION IN CASE THEY WON'T LET US USE THE ‘FIHDC BACTERIAL STRAIN’ LANGUAGE).

Nucleotide Sequence of Z0021:

(SEQ ID No. 1)
ATGAAGTGGCTTTTATTAATCACATTGAGTCTATATTCTTTTATTGTGC
AAAGTGCTCCCTGTGGATTGACCAATGTCGGGGAACAACGTGGAACATA
TATACTCAAACCACTGAGTATGAAAGGTAATCTCACCGCCGAAAAAATT
GAAAATACTGGGAATATCATTGAGTGTGCAGACTTGCAAGAGACGACCA
ACGCCATTAAAGTTCATTTTTTAACACAGAACGCTTTAAGGTTTAAAGT
CGGAAGTCGTCATTATATTATTAATTTCCCCTTTGAATCTGACGGTAAA
AAAAATAGCGCTTCCAGGGAAGTCATCTTATAA

Amino Acid Sequence of Z0021:

(SEQ ID No. 2)
MKWLLLITLSLYSFIVQSAPCGLTNVGEQRGTYILKPLSMKGNLTAEKI
ENTGNIIECADLQETTNAIKVHFLTQNALRFKVGSRHYIINFPFESDGK
KNSASREVIL

In one aspect of the invention, there is provided a method of reducing motility in a bacterium comprising: reducing surface expression of flagella in the bacterium by providing increased levels of a peptide having an amino acid sequence as set forth in SEQ ID No. 2, thereby reducing motility of the bacterium compared to a bacterium of similar type having normal levels of said peptide.

As will be appreciated by one of skill in the art, the bacterium may be a motile bacterium or may be a flagellated bacterium or may be a gram negative bacterium or may be a flagellated gram negative bacterium.

The levels of the peptide may be increased by introducing into the bacterium a nucleic acid molecule having a nucleotide sequence as set forth in SEQ ID No. 1. For example, a DNA molecule comprising a nucleotide sequence as set forth in SEQ ID No. 1 operably linked to a suitable promoter may be introduced into the bacterium by any suitable means known in the art, for example, by transformation, transfection or other suitable means of DNA transfer into a bacterium known in the art. In other embodiments, the expression of Z0021 may be increased by other means, for example, by increasing transcription.

As will be understood by one of skill in the art, ‘increased levels’ does not simply refer to copy number and/or total protein but also refers to increased activity. Thus, increased levels of Z0021 may in some embodiments refer to more active peptides, that is, peptides having greater repressor activity.

As will be appreciated by one of skill in the art, ‘normal’ levels may refer to wild type levels, for example, in a bacterium which has been transformed with a control sequence or mock transformed or is simply a ‘wild-type’ bacterium, that is, a bacterium in which no steps have been undertaken to increase the levels of Z0021.

### Deletion of OI-1 Results in Increased Motility of O157:H7 STEC

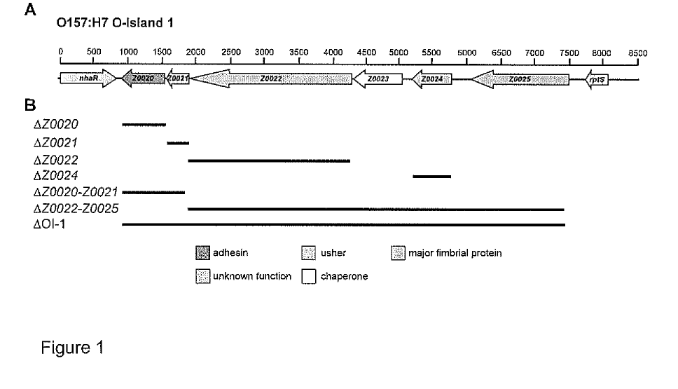

We previously showed that OI-1 is a genomic island unique to seropathotype A strains of Shiga toxin-producing E. coli (serotype O157:H7) and hypothesized that OI-1 may confer unique virulence or adaptive traits to seropathotype A strains. To test this idea, we created an isogenic deletion mutant of OI-1 in wild-type EDL933, which knocked out five genes coded in OI-1 (Z0021, Z0022, Z0023, Z0024, and Z0025), and examined the phenotype of the resulting mutant. To determine the effect that OI-1 deletion has on motility, the wild-type strain EDL933 and ΔOI-1 were evaluated for their motility in 0.25% LB agar. ΔOI-1 showed a significant increase in swimming at 25° C. and 37° C. The average diameter of swimming of ΔOI-1 was drastically higher (about 3 fold; p<0.0001) than that of wild-type EDL933 at 15 h and 6 h of incubation at 25° C. (data not shown) and 37° C. (FIG. 1A), respectively.

### Screening for Genes Involved in Repression of Motility in Wild-Type EDL933

The majority of E. coli strains including wild-type EDL933 are poorly motile (Ewing W H 1986). It is usually necessary to passage them serially through several rounds of semisolid agar motility medium to enhance motility and H antigen development. ΔOI-1 was highly motile and its swimming motility in 0.25% LB was prominent at 37° C. after 3 h incubation. To determine which specific gene on OI-1 was responsible for motility repression, we cloned each full-length OI-1 gene, (Z0021, Z0022, Z0023, Z0024, Z0025) and the entire OI-1 into an IPTG-inducible plasmid. Each complementation plasmid was transformed into ΔOI-1, and the strains were tested for their motility using 0.25% LB agar with or without 1 mM IPTG. An empty plasmid, pFLAG-CTC, was transformed into ΔOI-1 to serve as a control. Complementation of pZ0021, pZ0022, and pZ0024 in ΔOI-1 restored the less motility phenotype (FIG. 1B, 1C, p<0.0001). ΔOI-1 expressing pZ0023, pZ0025 or the empty plasmid pFLAG-CTC exhibited no significant change in motility (FIG. 1B, 1C). Surprisingly, complementation of pOI-1 did not restore the poor motility phenotype. We also constructed pZ0020, transformed into ΔOI-1 and tested for motility. Z0020 is positioned downstream of Z0021 and only part of Z0020 is in OI-1. Complementation of pZ0020 in ΔOI-1 significantly enhanced its motility (FIG. 1B, 1C, p<0.0001).

### Deletion of Z0021 but not Z0022-Z0025 Increased Motility in Wild-Type EDL933

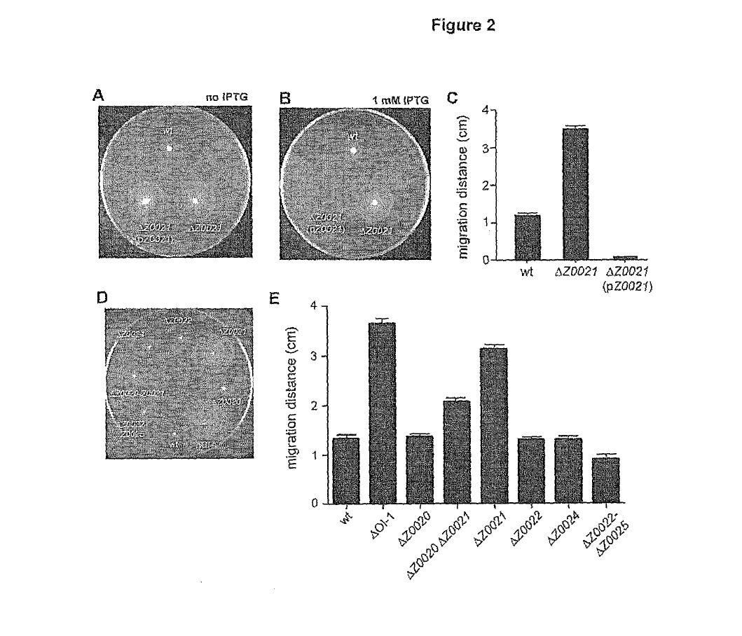

We determined that overexpression of Z0021, Z0022 and Z0024 restored ΔOI-1 motility to the wild-type motility phenotype. We would like to know further whether deleting each of these genes in the wild-type EDL933 would enhance the motility of the wild-type. We created Z0021, Z0022, Z0024 and Z0022-Z0025 mutants. Z0021 mutant (ΔZ0021) showed enhanced motility (FIG. 1B, 1C, p<0.0001). To verify that the specific deletion of Z0021 in EDL933 was responsible for the increased motility, we performed complementation analysis by transforming pZ0021 into the ΔZ0021 and tested for its motility. Overexpression of Z0021 readily suppressed motility of ΔZ0021 (FIG. 1B, 1C). Overexpression of proteins using an expression plasmid in bacteria may not represent an ideal situation for protein expression at physiological level. In order to confirm that Z0021 in vivo expression was responsible for repression of motility in the wild type, we deleted OI-1 genes (Z0022, Z0023, Z0024 and Z0025) except Z0021 (named ΔZ0022-Z0025). Motility of ΔZ0022-Z0025 was compared with the wild-type EDL933, ΔOI-1 and ΔZ0021. As we expected, ΔZ0022-Z0025, exhibited poor motility phenotype (FIG. 2D, 2E). Taken together, Z0021 is a motility repressor in wild-type EDL933.

### Deletion of OI-1 or Z0021 Enhanced Surface Expression of Flagella

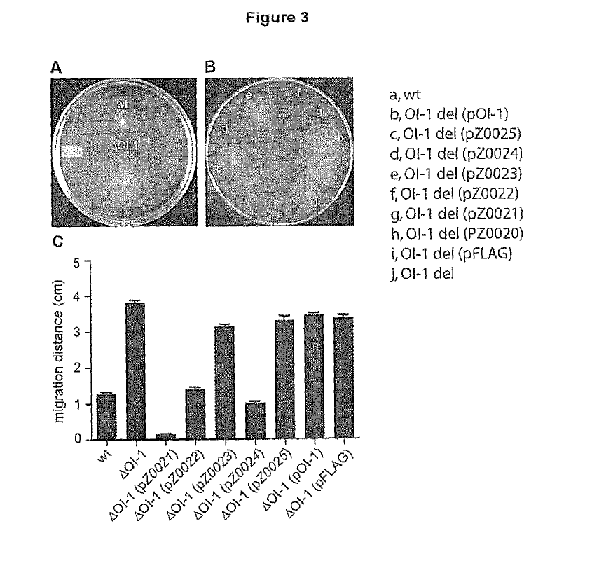

To distinguish whether the observed enhanced motility in ΔOI-1 and ΔZ0021 was due to an increase in flagella motility function or to an induction in flagellar biosynthesis, we examined the wild-type EDL933, ΔOI-1 and ΔZ0021 for their production of flagella by the Western blot analyses using H7 flagellin antiserum (FIG. 3A), and cellular morphology by transmission electron microscopy (FIGS. 3B to 3D). For western blot analysis, we cultured bacteria in liquid media because it was more practical to extract bacterial proteins. At 3, 6 and 24 h incubation at 37° C., the amount of flagellin produced in ΔOI-1 and ΔZ0021 was greater than that of the wild-type EDL933. Flagellin was not detectable in the wild-type EDL933 at 3 h incubation while significantly expressed in ΔOI-1 and ΔZ0021. At 3, 6 and 24 h ΔOI-1 showed higher levels of flagellin production than that of the wild-type EDL933 and ΔZ0021. These flagellin expression results coincided with the motility results. Therefore, these results indicate that Z0021 represses motility of the wild-type EDL933 by reducing surface production of flagella. For TEM examination, bacteria were harvested from motility plates after 6 h incubation at 37° C. The surface expression of flagella was visualized after negative staining using 0.1% uranyl acetate. The majority of the wild-type EDL933 appeared to be non-flagellated (FIG. 3B). Only few of them possessed 1-2 flagella. On the other hand, ΔOI-1 and ΔZ0021 showed a highly flagellated phenotype (FIG. 3C, 3D). The TEM observation strongly supports the indication that Z0021 represses motility of the wild-type EDL933 by reduction surface production of flagella.

### Overexpression of Z0021 Results in Decreased Motility in Other Enterobacteriaceae

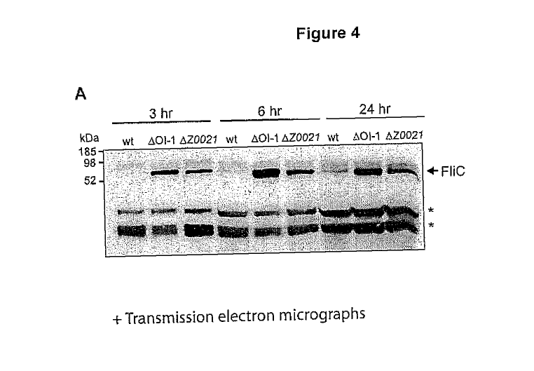

We investigated whether the flagellar repression effect of Z0021 was specific to E. coli O157:H7 strain EDL933. E. coli K12 strain MG1655 and S. Typhimurium strain SL1344 are highly motile bacteria in the Enterobacteriaceae family. To determine the effect of Z0021 on motility and surface flagellar expression, we overexpressed Z0021 by transforming pZ0021 into these bacteria and assayed for their motility. Overexpression of Z0021 in E. coli K12 strain MG1655 and S. Typhimurium strain SL1344 drastically reduced their motility in motility agar after incubation at 37° C. for 6 hr (FIG. 4A). Surface flagella were also assessed by TEM. While the wild-type E. coli K12 strain MG1655 and S. Typhimurium strain SL1344 were flagellated (FIG. 4D, 4F), their Z0021 complementation strains were rarely observed to produce flagella (FIG. 4E, 4G). Interestingly, more than 90% of E. coli K12 strain MG1655 expressing Z0021 showed membrane damage suggestive of toxicity (FIG. 4G′).

Bacterial strains and growth conditions. Bacterial strains and plasmids used in this study are listed in Table 1. E. coli O157:H7 strain EDL933, Salmonella enterica serovar Typhimurium, and isogenic mutants were propagated in Luria broth (LB) supplemented with antibiotics as appropriate. When used, antibiotic concentrations were (in μg ml⁻¹): ampicillin (Ap; 100); kanamycin (Kan; 50). Liquid cultures grown overnight were obtained by inoculating a single colony into LB containing appropriate antibiotics and incubation at 37° C. for 18 h with shaking (250 rpm). Bacterial strains were grown at 37° C., except for those containing the temperature-sensitive plasmid pKD46 (Ref), which was grown at 30° C. Primers were purchased from Laboratory Services, University of Guelph.

Construction of E. coli O157:H7 EDL933 in-Frame Deletion Mutants.

λ Red-mediated recombination was used to delete the entire OI-1, and individual genes within OI-1 (Z0021 to Z0025) in wild-type EDL933. Replacement of OI-1 or OI-1 gene with an antibiotic resistance cassette conferring resistance to kanamycin was achieved by integration of linear DNA containing a Kan resistance cassette flanked by FLP recognition sequences following transient expression of bacteriophage λ Red recombinase as described by Datsenko & Wanner (2000). PCR products used to construct gene replacements were generated using template plasmid pKD4 (Kan^{R}) and oligonucleotide primers listed in Table 2. The amplified products were concentrated on Qiagen spin columns, digested with DpnI for 3 hr at 37° C., then ethanol precipitated, and resuspended in DNase-free water, Wild-type EDL933 with plasmid pKD46 was induced with 100 mM L-arabinose for 2 h prior to harvest. Cells were electroporated with purified PCR products and plated on LB plates containing kanamycin (50 μg ml⁻¹).

### Motility Assay

Swimming motility was assessed using swimming agar plates containing 1% (w/v) tryptone, 0.5% NaCl and 0.25% agar (Difco Bacto Agar) with or without 1 mM IPTG (isopropyl-β-D-thiogalactopyranoside). A colony of bacteria was inoculated into 5 ml sterile LB broth with appropriate antibiotics and incubated at 37° C. with aeration (250 rpm) overnight. Overnight cultures were standardized to a McFarland standard of 1.0 (˜3×10⁸ cfu ml⁻¹) and 2 μl of the standardized cultures were stabbed into the agar plates using a sterile pipette tip. The plates were incubated for 6 h at 37° C. The diameter of the swimming zone around the inoculation site was measured and plates were photographed with Quantity One® (Bio-Rad).

### Complementation Plasmid Construction

To construct complementation plasmids, Z0021, Z0022, Z0023, Z0024 and Z0025 genes were PCR amplified separately using the primers listed in Table 1. The carboxy-terminal FLAG tag in our cloning strategy was eliminated by incorporation of the native stop codon from each gene. PCR products were cloned as HindIII/Bg/II fragments into similarly restriction-digested pFLAG-CTC (Sigma-Aldrich). This generated complementation plasmids with IPTG-inducible expression of the wild-type OI-1 genes designated pZ0021, pZ0022, pZ0023, pZ0024, pZ0025 and pOI-1.

### H7 Flagellin Protein Experiments

Western blot detection of H7 flagellin in wild-type EDL933, ΔOI-1 and ΔZ0021 was performed after diluting overnight cultures 1/100 in 5 ml of LB medium. Subcultures were grown with shaking at 250 rpm on a platform shaker for 3, 6 and 24 h at 37° C. Whole cell lysates were prepared for electrophoresis by adding an equal amount of 2× SDS sample buffer (100 mM Tris-HCl (pH 6.8), 20% (v/v) glycerol, 4% (w/v) SDS, 0.002% (w/v) bromophenol blue, 200 mM DTT), followed by boiling for 10 min. Samples were electrophoresed using discontinuous one-dimentional SDS-PAGE, and then transferred onto a polyvinylidene difluoride membrane (Immobilon-P. Milipore Corporation). The blots were incubated with 1:2000 dilution of rabbit polyclonal antiserum to H7 flagellin (Company), followed by a 1:2000 dilution of peroxidase conjugated mouse anti-rabbit immunoglobulin G, and developed using a chemiluminescent detection system (Pierce Chemical Company, Rockford, Ill., USA).

### Transmission Electron Microscopy

Wild-type EDL933, ΔOI-1 and ΔZ0021 were cultured in motility agar for 6 hr at 37° C. Bacteria were absorbed to carbon-stabilized Formvar supports on 200-mesh copper transmission electron microscopy (TEM) grids by floating the grid Formvar side down on a drop of culture for 30 sec, followed by a rapid wash with water. Bacteria on TEM grids were stained by submerging the grids for 10 sec in 0.1% (wt/vol) uranyl acetate and then examined with a Philips CM10 transmission electron microscope at an operating voltage at 80 kV. Digital images of bacteria were captured with a SIS/OLYPUS, Morada, 11 megapixel CCD camera (Biophysics Interdepartment Group, University of Guelph).

While the preferred embodiments of the invention have been described above, it will be recognized and understood that various modifications may be made therein, and the appended claims are intended to cover all such modifications which may fall within the spirit and scope of the invention.

### REFERENCES

Ewing W H 1986 Edwards and Ewing's Identification of Enterobacteriaceae, 4^{th} ed, Elsevier, N.Y.

TABLE 1
Bacterial strains and plasmids used in this study

| | | Reference or |
|---|---|---|
| Strain or plasmid | Genotype or description | source |
| Strains | | |
| E. coli | | |
| O157:H7 | Enterohemorrhagic E. coli serotype | Perna et al. |
| | O157:H7, strain EDL933 | |
| ΔOI-1 | EDL933 with deletion of O-island 1 | This study |
| | (ΔOI-1::Kan) | |
| ΔZ0021 | EDL933 ΔZ0021, Kn^{R} | This study |
| ΔZ0022 | EDL933 ΔZ0022, Kn^{R} | This study |
| ΔZ0024 | EDL933 ΔZ0024, Kn^{R} | This study |
| ΔZ0022-Z0025 | EDL933 ΔZ0022 ΔZ0022 ΔZ0024 ΔZ0025 | This study |
| E. coli K12 | MG1655, wild-type | ATCC 700926, |
| | | Blattner et al. |
| E. coli K12 (pFLAG- | MG1655, wild-type with plasmid pFLAG-CTC | This study |
| CTC) | | |
| E. coli K12 (pZ0021) | MG1655, wild-type with plasmid pZ0021 | This study |
| DH5α | hsdR recA lacZYA φ80 lacZΔM15 | ATCC |
| Salmonella enterica | | |
| sv. Typhimurium | | |
| SL1344 | SL1344, wild-type, Sm^{R} | Wray, C., and W. |
| | | J. Sojka. |
| SL1344 (pFLAG-CTC) | SL1344, wild-type, Sm^{r} with plasmid | This study |
| | pFLAG-CTC | |
| SL1344 (pZ0021) | SL1344, wild-type, Sm^{R} with plasmid | This study |
| | pZ0021 | |
| Plasmids | | |
| pFLAG-CTC | Cytoplasmic expression of C-terminal | Sigma-Aldrich |
| | FLAG fusion protein under control of the | |
| | Ptac, Ap^{R} | |
| pZ0021 | pFLAG carrying Z0021 with native stop | This study |
| | cod on | |
| pZ0022 | pFLAG carrying Z0022 with native stop | This study |
| | codon | |
| pZ0023 | pFLAG carrying Z0023 with native stop | This study |
| | codon | |
| pZ0024 | pFLAG carrying Z0024 with native stop | This study |
| | codon | |
| pZ0025 | pFLAG carrying Z0025 with native stop | This study |
| | codon | |
| pOI-1 | pFLAG carrying OI-1 | This study |
| pKD4 | oriRγ, Kan^{R} cassette flanked by FRT sites | Datsenko & |
| | (FLP recombinase target) | Wanner |
| pKD46 | RepA1019(Ts), λ, γ, and β exo expressed | Datsenko & |
| | from ParaBAD, Ap^{R} | Wanner |

TABLE 2
Oligonucleotides used in this study

| Oligo. | Sequence | Purpose |
|---|---|---|
| Red-Ol-1- | ggaatggtgaaatttatcgcagatagcatttcttctaaattataagatgtgtaggctgga | Ol-1 |
| F | gctgcttcg (SEQ ID No.3) | deletion |
| | | mutant |
| Red-Ol-1- | ctcataaattaaattaataaaaattcatcaacttcaaggactgataatatgaatgtectc | Ol-1 |
| R | ctta (SEQ ID No. 4) | deletion |
| Red- | cgatgaaaacaaaacatatatatgccagtaaaaggagtttactgtgtaggctggagct | Z0021 |
| Z0021-F | gcttcg (SEQ ID No. 5) | deletion |
| Red- | gtatggaatggtgaaatttatcgcagatagcatttcttctaaacatatgaatatcctc | Z0021 |
| Z0021-R | ctta(SEQ ID No. 6) | deletion |
| Red- | catagtaaaatcgccgcgagataacaggaaaaagtcgtgtaggctggagctgcttcg | Z0022 |
| Z0022-F | (SEQ ID No. 7) | deletion |
| Red- | tgtgattaataaaagccacttcatagtaaactccttcatatgaatatcctcctta | Z0022 |
| Z0022-R | (SEQ ID No. 8) | deletion |
| Red- | gagtgaccttccataccagttaattaaggatgatttgtgtaggctggagctgcttcg | Z0024 |
| Z0024-F | (SEQ ID No. 9) | deletion |
| Red- | aaataataccggtagcagagctaccggcaaaaaacgcatatgaatatcctcctta | Z0024 |
| Z0024-R | (SEQ ID No. 10) | deletion |
| Red- | taaggctaacctgactgcacagatcaacaaactggcttaagtgtaggctggagctgct | Z0022- |
| Z0022-25- | tcg (SEQ ID No. 11) | Z0025 |
| F | | deletion |
| Red- | taaaagaatatagactcaatgtgattaataaaagccacttcacatatgaatatcctcctta | Z0022- |
| Z0022-25- | (SEQ ID No. 12) | Z0025 |
| R | | deletion |
| Ol-1F | catgaagcttatgacagatggtatctcaac (SEQ ID No. 13) | Ol-1 |
| Ol-1R | cgcgagatcttagttgtacattaccac (SEQ ID No. 14) | Ol-1 |
| Z0020F | catgaagcttatggcgataaatttcacc (SEQ ID No. 15) | Z0020 |
| Z0020R | cgcgagatcttagttgtacattaccac (SEQ ID No. 16) | Z0020 |
| Z0021F | catgaagcttatgaagtggcttttatta (SEQ ID No. 17) | Z0021 |
| Z0021R | cgcgagatcttataagatgacttccct (SEQ ID No. 18) | Z0021 |
| Z0022F | catgaagcttatgttaaggacgacacga (SEQ ID No. 19) | Z0022 |
| Z0022R | cgcgagatcttactggcatatatatgt (SEQ ID No. 20) | Z0022 |
| Z0023F | catgaagcttgtgagaataataacagc (SEQ ID No. 21) | Z0023 |
| Z0023R | cgcgagatcttatctcgcggagattttac (SEQ ID No. 22) | Z0023 |
| Z0024F | catgaagcttatgaaaaagtatattattc (SEQ ID No. 23) | Z0024 |
| Z0024R | cgcgagatcttaatctgcactgatttt (SEQ ID No. 24) | Z0024 |
| Z0025F | catgaagcttatgacagatggtatctcaac (SEQ ID No. 25) | Z0025 |
| Z0025R | cgcgagatcttaatcttgatgagatat (SEQ ID No. 26) | Z0025 |

1. A method of reducing motility in a bacterium comprising:
reducing surface expression of flagella in the bacterium by providing increased levels of a peptide having an amino acid sequence as set forth in SEQ ID No. 2, thereby reducing motility of the bacterium compared to a bacterium of similar type having normal levels of said peptide.

2. The method according to claim 1 wherein the levels of the peptide are increased by introducing into the bacterium a nucleic acid molecule having a nucleotide sequence as set forth in SEQ ID No. 1.
